(19) United States
(12) Patent Application Publication
(10) Pub. No.: US 2013/0039978 A1
(43) Pub. Date: Feb. 14, 2013

(54) MEDICINAL COMPOSITIONS AND METHOD FOR TREATMENT OF URINARY TRACT INFECTIONS

(71) Applicants: Joseph Schwarz, Toronto (CA); Michael Weisspapir, Toronto (CA); Vedran Hasanagic, Kitchener (CA)

(72) Inventors: Joseph Schwarz, Toronto (CA); Michael Weisspapir, Toronto (CA); Vedran Hasanagic, Kitchener (CA)

(21) Appl. No.: 13/570,789

(22) Filed: Aug. 9, 2012

Related U.S. Application Data
(60) Provisional application No. 61/521,491, filed on Aug. 9, 2011.

(51) Int. Cl. A61K 36/61; A61K 36/537; A61P 13/02; A61K 9/48; A61K 36/00; A61K 36/752; A61K 36/899

(52) U.S. Cl. 424/456; 424/742; 424/736; 424/746; 424/745; 424/725

(57) ABSTRACT

The invention describes medicinal compositions, comprising combination of essential oils, and method for treatment of cystitis and urinary tract infections by oral administration of such compositions.

This application claims the benefit under 35 U.S.C. 119(e) of U.S. provisional application 61/521,491 tiled Aug. 9, 2011. which application is herein incorporated by reference in its entirety.

### BACKGROUND OF THE INVENTION

Cystitis (inflammation of the bladder) and other urinary tract infections (e.g., urethritis or pyelonephritis) usually are caused by pathogenic bacteria. Escherichia coli is the most common bacterium isolated and accounts for about 80% of community acquired Urinary Tract Infections (UTI). Among patients aged 20 to 50 years, UTI are about 50-folds often in women than in men. The standard treatment of acute cystitis includes antibacterial therapy. Fluoroquinolones, Trimethoprim-Sulfametoxazole or macrolide antibiotics usually effectively eradicate bacterial pathogens from urinary tract reservoirs. Nevertheless, the recurrence rate for UTI is high, and many women (up to 30%) have 3-5 UTI episodes per year.

Repeated courses of antibiotics can lead to increased bacterial resistance and also can cause dangerous adverse reactions. Additionally, inflammation of urinary tract is a painful and often debilitating symptom of bacterial infection. Obviously there is a demand for effective alternative medications (e.g., non-antibiotics) that can be used to treat or prevent urinary tract infections in both animals and humans.

Traditional treatment of UTI using natural remedies includes diuretics (Uva Ursi leafs, Polygonum (knot grass), plantain, juniper berries), plant antiseptics (e.g., cranberry juice or extract, containing benzoic acid and flavonoids [U.S. Pat. No. 7,270,837 “Anti-inflammatory cranberry flavonol extract preparations” to Vorsa et al., Sep. 18, 2007] are widely recommended but demonstrate no significant success. D-Mannose, introduced last decade as a remedy for cystitis via prevention of bacteria adhesion to bladder walls is more helpful but cure rate is still not as high as desired [Hagbarg L., pp. 566-569].

Thus, there is an unmet need for non-antibiotic treatments for urinary tract infections. There is also a need for safe and effective method for treatment and prevention of cystitis, which can be conveniently used for self-medication.

We have surprisingly discovered that combination of several essential oils of the present invention, given orally, demonstrates superior activity against urinary tract infections. These compositions are suitable for use in the treatment of UTI disorders, particularly those of the urinary tract arising from bacterial sources, especially E.coli. Moreover, these compositions can be formulated to contain combination of essential oils that can inhibit the invasion of uroepithelial cells by different types of E. coli and prevent adhesion of the bacteria to bladder. Advantageously, these compositions can be administered, in formulations, where irritant nature of the essential oils to the digestive mucous membranes can be successfully suppressed, eliminating adverse effects caused by oral administering of these oils. Thus, the proposed compositions of the present invention represent safe and effective natural treatment that can he used beneficially for self-medication by those afflicted with urinary tract infections, particularly cystitis.

Few publications describe prevention of bacterial biofilm formation [Budzy\check{n}ska A., et al., 2011] or bactericidal effect of volatile mustard oil and herbal extract [Conrad A., et al., 2006], but the biological activities were investigated only “in vitro”. Or use of essential oils for treatment of UTIs has never been mentioned or described in literature before. The US Patent Application 20100136207 describes preparation of nanoemulsions containing low levels (0.03-0.3% of spice essential oils from oleoresins extracts. Said nanoemulsions were prepared by high pressure homogenization in presence of biopolymers and high concentration of surfactants and designed for use as flavour additives in food industry.

### DESCRIPTION OF THE INVENTION

Extract of Juniper berries is well known as potent diuretic and recommended for use in some methods of UTI treatment. Nevertheless, there were no attempts to replace of alcoholic extract to more concentrated essential oil. Moreover, essential oils mentioned as remedies for different diseases and conditions, including cystitis, but only as aromotherapeutic ingredients or components of topical applications, for bath or massage applications [Worwood, V. A. “The complete book of essential oils and aromatherapy” New World Library Novato Calif. 1991, page 252].

Oral delivery of some essential oils, given in relatively high concentrations, can significantly improve symptoms and cure UTI, especially cystitis, either acute or chronic. Surprisingly we found that essential oils in combination work more efficiently than any of the oils, used separately, i.e., mixture of several oils demonstrating pronounced synergism between components.

Numerous volatile oils, such as eucalyptus oil, lavender oil, sage oil, pine oil, fir needle oil, cedar oil, turpentine oil, cajeput oil, bergamot oil, rosemary oil, oregano oil, celery oil, sandalwood oil, lemongrass oil, cypress oil, juniper oil, bay laurel oil, grapefruit oil, thyme oil and tea tree oil were tested trying to treat UTI. Separately used, juniper oil, bergamot oil, rosemary oil, cypress oil, eucalyptus oil, lavender oil, pine oil, and cajeput oil have a visible diuretic activity being given orally on a piece of sugar or in alcohol-water mixture. But several mixtures of the 2-4 oils, combined together in various ratios, demonstrated higher diuretic activity in lower doses and also revealed fast and apparent suppression of cystitis symptoms. For some combinations cystitis symptoms were eliminated in 24-72 hours after first oral administration of the essential oils mixtures; usually mixtures were given in small amounts, 0.1-05 ml (calculated as sum of essential oils) 2-3 times a day.

Due to a strong acrid taste of many of the essential oils they can be administered in appropriate capsules (sealed hard or soft gelatin capsules). Also some of the mixtures can be delivered being dissolved in water miscible physiologically acceptable solvent. Concentrated ethyl alcohol is the solvent of choice because solubility of these oils in other water miscible solvents (e.g., propylene glycol, liquid polyethylene glycol, ethoxydiglycol, dimethylisosorbid, glycerin) is limited. Solution of the it combinations in alcohol can be diluted before use by mixing with a drink (water, juice).

### EXAMPLES 1-6

### Essential Oil Mixtures for Oral Administration in Gelatine Capsules (Amounts Per Single Capsule, mg)

| Component | Ex. 1 | Ex. 2 | Ex. 3 | Ex. 4 | Ex. 5 | Ex. 6 |
|---|---|---|---|---|---|---|
| Lavender oil | 50 | 50 | 100 | 50 | | 80 |
| Rosemary oil | 100 | | | | 100 | |
| Sage oil | 100 | | | 200 | | |
| Turpentine oil | | 50 | | 50 | | 80 |
| Eucalyptus oil | 100 | | 200 | | | 100 |
| Juniper oil | | | 100 | 50 | 100 | 100 |
| Cajeput oil | | | | | 50 | |
| Bergamot oil | | 50 | | | | |
| Pine oil | | 50 | | | 100 | |
| Olive oil | | to 400 mg | to 400 mg | | | |
| Capric/Caprylic | | | | to 400 mg | | |
| triglycerides | | | | | | |

All components were accurately weighed, mixed and transferred to hard gelatine capsules (#00). Capsules were sealed using hot solution of gelatin. Same compositions can be prepared in soft gelatine capsules. Addition of glyceride oils helps to improve compatibility with material of capsule walls.

### EXAMPLES 7-12

### Essential Oil Mixtures for Oral Administration in Alcohol Solution (Amounts Per 50 mL of Final Solution, mL)

| Component | Ex. 7 | Ex. 8 | Ex. 9 | Ex. 10 | Ex. 11 | Ex. 12 |
|---|---|---|---|---|---|---|
| Eucalyptus oil | 1.5 | | | 1.4 | 1.6 | 1.5 |
| Turpentine oil | | 0.5 | | | 1.0 | 0.5 |
| Cypress oil | | | 1.0 | | | |
| Bay laurel oil | | | | 0.6 | | |
| Lavender oil | 0.2 | 0.5 | | 0.8 | 1.1 | 0.5 |
| Juniper oil | 1.5 | | 1.5 | | 1.6 | 1.5 |
| Lemongrass oil | | 1.0 | 0.3 | | | 0.6 |
| Grapefruit oil | | | | 2.2 | | |
| Thyme oil | 0.3 | 1.0 | 0.2 | | | 0.4 |
| Ethyl alcohol | To | To | To | To | To | To |
| USP 95% | 50 mL | 50 mL | 50 mL | 50 mL | 50 mL | 50 mL |

Selected essential oils were combined in an appropriate vessel, then alcohol was added while mixing and formed clear solutions stored tightly closed. For oral administration formulations 7-12, were added to drinking water (0.5-1 ml of the formulation per 50-100 ml of water), shaken or stirred and consumed immediately. This way allows to provide exact dosing of the essential oils according, to bodyweight of the person in single portion. The undesired experience is that after dilution of alcoholic solution the layer of essential oils floats on the water surface, thus providing unpleasant taste, also a significant part of the oils adheres to the surface of the drinking glass and stirring spoon or spatula.

Essential Oil Mixtures for Oral Administration in Self-Emulsifying Vehicle

To eliminate the observed problems with taste and adherence of essential oil compositions several formulations were incorporated into self-emulsifying delivery systems, which forms oil-in-water emulsion after contact with water containing media, and where essential oils remains in the oil phase of the emulsion. In this case there is no oil phase separation and floating, and the taste of essential oils is masked by inclusion in the hydrophobic matrix. Moreover, it was found that when submicron emulsion (nanoemulsion) is formed, absorption of the essential oils in gastro-intestinal tract is fast and complete, and biological activity of the oils significantly increases. During preparation we found that emulsification of pure essential oils is a complicated process and requires very high concentrations of surfactants, which is questionable from the physiological point of view. Surprisingly it was discovered that addition of even small amounts of some hydrophobic materials, chosen in accordance with physico-chemical properties of the essential oils, allowed us to prepare easy and smooth self-emulsifying compositions. These hydrophobic compounds, such as relatively polar aliphatic lipids capric/caprylic triglycerides, acetylated mono- and diglycerides) or aromatic compounds (e.g., liquid ester of tocopherol) permitted the amount of surfactants required for self-emulsification to be significantly decreased. Addition of alcohol accelerates self-emulsifying process and decrease viscosity of the composition, making mixing process faster and more efficient.

### EXAMPLES 13-18

### Essential Oil Mixtures in Self-Emulsifying Compositions for Oral Administration (Amounts Per 50 mL, Weight/Volume)

| Component | Ex. 13 | Ex. 14 | Ex. 15 | Ex. 16 | Ex. 17 | Ex. 18 |
|---|---|---|---|---|---|---|
| Lavender oil | 4.0 | 1.5 | | | 1.5 | 1.2 |
| Rosemary oil | | 1.0 | 2.0 | | 1.5 | |
| Sage oil | | | 2.0 | | | |
| Turpentine oil | | | 1.0 | 1.0 | | 0.8 |
| Eucalyptus oil | 2.0 | | | | 2.5 | |
| Juniper oil | 4.0 | 2.0 | 2.0 | 2.5 | | 1.8 |
| Tea tree oil | 2.0 | | | | | |
| Bergamot oil | | | 0.5 | | | |
| Sandalwood oil | | | | | 1.0 | |
| Fir needle oil | 3.0 | 2.5 | | | | 1.2 |
| Olive oil | | 10 | | | 10 | |
| Capric/Caprylic triglycerides | | | 5.0 | | | 10 |
| Tocopherol acetate (Vitamin E) | | | | 2.0 | 0.4 | 0.5 |
| Acetylated mono-diglycerides | | 18 | 12 | 15 | 10 | 6.0 |
| Lecithin 75% | | | | | 2.0 | 3.0 |
| Distearoylphosphatidylcholine | | | | 3.0 | | |
| Polysorbate 20 | 25 | | | 5.0 | | 8.0 |
| Polysorbate 40 | | 10 | | | | |
| Polysorbate 60 | 10 | | | 4.0 | | 4.0 |
| Polysorbate 65 | | 5.0 | | | | |
| Polysorbate 80 | | | | | 8.0 | |
| Polysorbate 85 | | | | | 4.0 | |
| Polyoxyl 35 castor oil | | | 8.0 | | | |
| PEG 40 stearate | | | 6.0 | | | |
| Ethyl alcohol | | | to 50 mL | to 50 mL | to 50 mL | to 50 mL |
| Self-emulsification efficacy | + + + | +− | + + | + + | + | + + + |

### EXAMPLES 19-24

### Essential Oil Mixtures in Self-Emulsifying Compositions for Oral Administration (Amounts Per 50 mL, Weight/Volume)

| Component | Ex. 19 | Ex. 20 | Ex. 21 | Ex. 22 | Ex. 23 | Ex. 24 |
|---|---|---|---|---|---|---|
| Lavender oil | 2.0 | | 2.0 | 2.0 | 1.5 | 1.2 |
| Rosemary oil | | | | | 1.5 | |
| Sage oil | 1.5 | | | | | |
| Turpentine oil | | | 3.0 | | | |
| Eucalyptus oil | | | | 2.2 | 1.2 | 2.0 |
| Juniper oil | 2.0 | 1.6 | 1.5 | 1.8 | 2.0 | 1.6 |
| Tea tree oil | 1.2 | | | | | |
| Bergamot oil | | 1.0 | | | | |
| Sandalwood oil | | | 2.0 | | | |
| Fir needle oil | | | | 1.0 | 1.5 | 1.2 |
| Capric/Caprylic triglycerides | 15 | | 10 | | | |
| Tocopherol acetate (Vitamin E) | | 2.0 | 5.0 | | 4.0 | 3.0 |
| Acetylated mono-diglyceridcs | | 12 | | 12 | 14 | 15 |
| Lecithin 75% | 5.0 | 2.5 | 3.0 | | 2.5 | 3.0 |
| Distearoylphosphatidylcholine | | | | 2.0 | | |
| Polysorbate 20 | | 5.0 | | | 6.0 | |
| Polysorbate 60 | | 5.0 | | | 2.0 | |
| Polysorbate 80 | | | 6.0 | | | |
| Polyoxyl 40 hydrogenated | | 6.0 | | 8.0 | | 5.0 |
| castor oil | | | | | | |
| PEG 40 stearate | | 4.0 | | 2.0 | | 1.5 |
| Poloxamer ™ 188 | 10 | | | | | |
| Sucrose stearate | | | 4 | | | |
| Ethyl alcohol | to 50 ML | to 50 mL | to 50 mL | to 50 mL | to 50 mL | to 50 mL |
| Self-emulsification efficacy | − | + + + | + | + + + | + + + | + + + |

Preparation: Selected combination of essential oils, hydrophobic components, surfactants and alcohol were combined according to proposed ratio in an appropriate vessel. The vessel was tightly closed and shaken for 5-15 minutes until clear solution was formed.

For investigation of self-emulsifying behavior 0.5 ml of a formulation was added at room temperature to 50 mL of pure water while stirring with a stainless steel spatula. Observations are presented in tables for examples 13-18 and 19-24. (“−” relates to had or no emulsification; “+−—” poor emulsification; “+”, “++” and “+++” correspondently mean fair, good and excellent emulsification.

Formulation 13, comprising mixture of essential oils and surfactants forms micellar solution when combined with water. The adherence of the essential oils to glass surfaces and steel was after dilution was significantly decreased. This composition has very high level of surfactant and does not contain additional hydrophobic phase, and due to extremely small size of the formed micelles the taste masking was not so good. Addition of the oil components in composition 14 lead to emulsification, but for this combination of oils and surfactants formed emulsion was coarse and unstable. Nevertheless, even such coarse emulsion demonstrated good taste and smell masking of the incorporated essential oils. Addition of ethanol also accelerates emulsification and improved properties of the formed emulsion. Products containing phospholipids (lecithin, distearoylphosphatidylcholine) as a co-surfactant tend to form emulsion with small droplet size, usually below 500 nm. Use of relatively polar oils (capric/caprylic triglycerides, acetylated mono- and diglycerides, tocopherol acetate) separately or in combination helps to obtain stable oil-in-water emulsion with relatively low content of surfactants. Combinations of essential oils with oil having low polarity (e.g., olive oil) are more difficult to emulsify. Average size of the oil droplets in most of tested emulsions, obtained by self-emulsifying, varies from 70 to 300-500 nm, with relatively narrow size distribution.

In order to provide a formulation with satisfactory organoleptic properties, additional compounds such as sweeteners, pH stabilizers, colours and flavors can be added. Sometimes it can infringe self-emulsifying process and cause instability of the formed emulsion, so a careful adjustment of composition may require.

### EXAMPLES 25-31

### Essential Oil Mixtures in Self-Emulsifying Compositions for Oral Administration (Percent Composition)

| Component | Ex. 25 | Ex. 26 | Ex. 27 | Ex. 28 | Ex. 29 | Ex. 30 | Ex. 31 |
|---|---|---|---|---|---|---|---|
| L-Menthol crystalline USP | | | 0.5% | | | | |
| Peppermint oil USP | | | | | | 0.7% | |
| Lemon oil | | | | | | | 1.0% |
| Orange oil | 0.71% | | | | | | |
| Juniper oil | 3.4% | 3.5% | 2.0% | 3.5% | 3.5% | 3.0% | 6.2% |
| Lavender oil | 2.3% | 2.32% | 4.0% | 2.3% | 2.3% | 6.0% | 4.1% |
| Eucalyptus oil | 2.3% | 2.90% | 4.0% | 2.9% | 2.9% | 6.0% | 4.1% |
| Fir needle oil | 2.3% | 2.32% | 2.0% | 2.3% | 2.3% | 3.0% | 4.1% |
| Acetylated mono- and | 35.3% | 31.9% | 14.4% | 32.1% | 31.9% | 18.0% | |
| diglycerides | | | | | | | |
| Capric/caprylic triglycerides | | | | | | | 20.5% |
| Tocopherol acetate (Vitamin E) | 4.2% | 6.1% | | 5.8% | 6.1% | 3.0% | 3.1% |
| Lecithin 75 | 7.06% | 7.24% | 3.0% | 7.4% | 7.3% | 4.50% | 5.14% |
| Sucralose | | 0.22% | 0.50% | 0.2% | 0.22% | 0.20% | |
| Glycirrhysic acid ammonium salt | | | | | | 1.0% | |
| Neothame | | | | | | | 0.05% |
| Saccharine | 0.14% | 0.14% | | | | | |
| Ethyl alcohol | 28.25% | 28.96% | 64.10% | 31.4% | 29.0% | 44.6% | 41.1% |
| Citric acid | | | | | | 0.50% | 0.31% |
| Sodium citrate | | | | | | 0.25% | |
| PEG stearate | 2.8% | | | | 2.9% | 2.25% | 2.1% |
| Polysorbate 60 | | 2.9% | | 2.6% | | | |
| Polyoxyl 40 hydrogenated | 11.30% | | 4.0% | | 11.6% | 6.0% | 8.2% |
| castor oil | | | | | | | |
| Polysorbate 20 | | 11.6% | 1.5% | 9.4% | | | |
| Self-emulsification efficacy | + + + | + ++ | + + + | + + + | + + + | + + + | + + + |

Preparation: Selected combination of essential oils, hydrophobic components, surfactants, flavors and alcohol were combined according to proposed ratio in an appropriate vessel. The vessel was tightly closed and shaken for 5-15 minutes until clear solution was formed. Sweeteners and buffering components were added to a mixture and shaken or stirred in a closed vessel for 30-90 minutes.

Formulations 19-31, being mixed with water at room temperature (0.5 mL per 50 mL of water), immediately forms oil-in-water emulsions, stable for at least 2 hours. The formed dispersion has a pleasant taste; the smell of essential oils is masked. These emulsions can easily be consumed by patients. No stomach irritation or other undesirable side effects were observed.

Oral Administering to Patients in Need

Multiple formulations (##5, 11, 13, 18, 22, 29, etc.), comprising essential oils in different vehicles according to the invention, were delivered to number of patients with diagnosed cystitis or symptoms of UTI.

R. G., female, 38 years old. Diagnosed with chronic cystitis for 3.5 years. Had numerous antibiotic courses in the previous history. Treated with oral administration of the encapsulated composition eliminated all symptoms of the cystitis in 5-7 days.

I. D., female. 31 years old, Recurrent chronic cystitis. E. coli counts in urine 10×10⁶/mL. Treated orally with diluted liquid formulation (3 times×1.0 mL a day for 21 days). Urine sterile.

L. A., female, 3 years old. Cystitis, resistant to antibiotics. E. coli counts in urine 9×10⁶. Treated orally with diluted liquid formulation (3 times×0.15 mL a day for 28 days). Urine sterile.

D. U., female, 49 years old. Chronic cystitis for more than 5 years. E. coli counts in urine 1×10⁶/mL. Treated orally with diluted liquid formulation (3 times×1.5 mL a day for 7 days). All symptoms disappeared. E. coli counts<10³/mL.

V. D., male, 71 years old. Acute urinary tract infection, bacterial counts 10×10⁶/mL. Patient allergic to antibiotics. Treated orally with diluted liquid formulation (3 times×1.2 mL a day for 14 days). Symptoms disappeared, urine sterile.

A. M., female, 56 years old. Cystitis, resistant to antibiotic treatment. E.coli counts in urine 10×10⁶/mL. Treated orally with diluted liquid formulation (3 times×mL a day for 21 days). Symptoms disappeared, E. coli counts<10⁴/mL.

M. P., male, 9 years old. Recurrent UTI, resistant to antibiotics. Treated orally with diluted liquid formulation (3 times×0.2 mL a day for 28 days). Symptoms disappeared, urine sterile.

### REFERENCES

U.S. Pat. No. 7,270,837 Vorsa N, et al., “Anti-inflammatory cranberry flavonol extract preparations” Sep. 18, 2007, col. 1 lines 31-67, col. 2 lines 11-67, col. 3 lines 54-67, col. 4 lines 1-45, col. 5 lines 30-67, col. 6 lines 1-45

United States Patent Application 20100136207 “NANOEMULSION AND NANOPARTICLE CONTAINING PLANT ESSENTIAL OIL AND METHOD OF PRODUCTION THEREOF”, pages 2, 3 paragraphs 007-024.

HAGBERG, L., et. al “Adhesion, Hemagglutination, and Virulence of Escherichia coli Causing Urinary Tract Infections” INFECTION AND IMMUNITY, February 1981. p. 564-570, Vol. 31, No. 2

WORWOOD, Valerie Ann “The complete book of essential oils and aromatherapy” New World Library Novato Calif. 1991 page 252

Budzy\check{n}ska A, Wieckowska-Szakiel M, Sadowska B, Kalemba D. Rózalska B. “Antibiofilm activity of selected plant essential oils and their major components”. Pol. J. Microbial. Vol. 60 No. 1, pp. 35-41. (2011)

Conrad A, Kolberg T. Engels I, Frank U. “In vitro study to evaluate the antibacterial activity of a combination of the haulm of nasturtium (Tropaeoli majoris herba) and of the roots of horseradish (Armoraciae rusticanae radix)”. Arzneimittelforschung. Vol. 56 No. 12 pp. 842-849. (2006)

1. An orally administered composition for treatment of cystitis and urinary tract infections, comprising a mixture of at least two essential oils, selected from group of eucalyptus oil, lavender oil, sage oil, pine oil, fir needle oil, cedar oil, turpentine oil, cajeput oil, bergamot oil, rosemary oil, oregano oil, celery oil, sandalwood oil, lemongrass oil, cypress oil, juniper oil, bay laurel oil, grapefruit oil, thyme oil and tea tree oil.

2. A composition of claim 1 comprises mixture of eucalyptus oil, lavender oil, fir needle oil and juniper oil.

3. A mixture of essential oils of claim 1, wherein said mixture comprises of 2-60% of eucalyptus essential oil, 2-60% of lavender essential oil, 2-60% of fir needle oil or pine oil and 2-60% of juniper essential oil.

4. A mixture of essential oils of claim 1 comprising juniper oil, lavender oil, eucalyptus oil and fir needle oil in ratio from 3:2:2.5:2 to 4:0.5:1:1.

5. A composition of claim 1 for oral administration, wherein said mixture of essential oils administered in a soft gelatine capsule.

6. A composition of claim 1 for oral administration, wherein said mixture of essential oils administered in a sealed hard gelatine capsule.

7. A composition of claim 1 for oral administration, wherein said mixture of essential oils administered dissolved in alcohol.

8. A composition of claim 1 for oral administration of essential oils, wherein said mixture of essential oils administered in self-emulsifying composition.

9. A self-emulsifying composition of claim 8, comprising of
a. 2 to 60% of mixture of essential oils,
b. 0.5 to 95% of a physiologically acceptable hydrophobic material to dissolve essential oils,
c. 0.5-50% of and physiologically acceptable surfactant or mixture of surfactants,
d. 0.05-80% of alcohol,
and said self-emulsifying composition forms oil-in-water emulsion when contacted with water containing media.

10. A composition of claim 9, wherein said hydrophobic material is selected from the group of capryc/caprylic triglycerides, acetylated mono/diglycerides and tocopherol acetate.

11. A composition of claim 9, comprising mixture of surfactants with composite HLB of the mixture in the range of 3 to 18.

12. A self-emulsifying composition of claim 11, comprising a polysorbate or an ethoxylated castor oil.

13. A composition of claim 12, comprising a polysorbate and a phospholipid.

14. A composition of claim 12, comprising poly hydrogenated castor oil and lecithin or phosphatidylcholine.

15. A composition of claim 13, comprising polysorbate-20, polysorbate-60 and soy lecithin.

16. A self emulsifying pharmaceutical composition of claim 9, comprising of 0.5-10% of eucalyptus oil, 0.5-10% of lavender oil, 0.5-10% of fir needle oil, 0.5-10% of juniper oil, 2-50% of acetylated mono/diglycerides, 0.5-10% of tocopherol acetate, 2-20% of polysorbate-20, 2-20% of polysorbate-60, 1-10% of lecithin or phosphatidylcholine and 10-90% of alcohol.

17. A self emulsifying pharmaceutical composition of claim 9, which may additionally comprise physiologically acceptable sweeteners, preservatives, flavors and colorants

18. A method for the treatment of cystitis and urinary tract infections or for the protection against such diseases involving the step of administering to a patient in need an effective, amount of the mixture of essential oils, selected front group of eucalyptus oil, lavender oil, pine oil, fir needle oil, turpentine oil and juniper oil.

19. A method for the treatment of cystitis and urinary tract infections as of claim 18, comprising oral administering of self-emulsifying composition of essential oils mixture in dose of 0.01-1.0 g of said essential oil mixture 1-3 times a day for 5-30 days.
